(19) United States
(12) Patent Application Publication
(10) Pub. No.: US 2007/0254284 A1
(43) Pub. Date: Nov. 1, 2007

(54) A nucleic acid detection method

(76) Inventor: Biwei Zhao, Collegeville, PA (US)

Correspondence Address: BIWEI ZHAO, 3815 VINCENT DR., COLLEGEVILLE, PA, 19426, US

(21) Appl. No.: 11/381,069

(22) Filed: May 1, 2006

(51) Int. Cl. C12Q 1/68

(52) U.S. Cl. 435/6

(57) ABSTRACT

A nucleic acid detection method utilizing fluorescent reporter for real-time monitoring of amplification in a solid state where both forward and reverse primers are attached to a array with a plurality of primers.

### BACKGROUND

The technology of repeatedly amplifying nucleic acids in a sample, the polymerase chain reaction (PCR), has greatly broadened our capabilities of studying biological processes. There is a great deal of new developments that are based on the concept of conventional PCR. Among them is quantitative PCR, or real-time PCR, which utilizes fluorescent reporters to measure the progress of the amplification (real-time monitoring), versus measuring amplification at the end of the reaction in a conventional PCR (end-point measuring). The amplification in a PCR typically has a sigmoidal growth curve. In the middle of this sigmoidal growth curve is a log-linear phase where the accumulation of the amplicon best reflects the original amount of the target. In a conventional PCR the end-point measurement will usually miss this log-linear phase, therefore it does not correctly represent the initial amount of the target. Only in real-time PCR where the log-linear phase is recorded, the original amount of the target can be determined.

Another novel strategy in biological research is so-called global study. This new concept intends to study multiple genes, not just one or a few genes, at the same time. One good representative of this global study strategy is the DNA microarray chip technology where multiple copies of different oligonucleotide probes are attached to a solid surface, the chip, and this chip is hybridized with samples that are pre-amplified and labeled with fluorescent reporters. There are two samples, the reference and the study sample, in a typical microarray assay. One sample is usually labeled with green fluorescent dye and the other sample with red fluorescent dye. Samples are amplified to ensure sufficient amount needed for generating enough signals for detecting. These two labeled samples are mixed in equal amount and hybridized with the microarray chip. Detecting of the fluorescent color (more green or more red) will indicate which gene is more abundant in which sample.

The microarray chip technology has lots applications. One of them is called expression profiling. In a cell RNA fragments are transcribed using genomic DNA as a template. These transcribed RNA fragments are called transcripts. Biological researches have found that the amount of transcripts vary greatly, representing activation or inhibition of certain genes in normal cells and in diseased cells such as cancerous cells. Therefore the expression profiling can detect which genes are activated or inhibited in which cells. In a typical expression profiling, the total RNA isolated from two samples, such as from normal cells and from tumor cells, are first reverse transcribed, which yield a pool of cDNAs. These cDNA pools are then amplified and labeled with either green fluorescent dye or red fluorescent dye. They are then mixed and hybridized with the microarray chip that contains oligonucleotide probes for hundreds of thousands of genes.

A much scaled-up expression profiling is called a tiling array. In a tiling array the probes are generated in such a density that there is one probe for every 100 bases or so along the whole genome, or a substantial portion of the genome. This allows a genome-wide global study, which is now critical in such topics as identification of disease-related genes and drug discovery. This is not achievable until high density microarray chips, with up to several millions of features on a single array, become available. This tiling array enables detection of rare transcripts that are missed in other low-density arrays.

Another application of tiling array is for study of single nucleotide polymorphism (SNP). SNP detections are a method of genotyping, the identification of the genetic characteristics of a disease or an individual. SNP detections have been broadly used in drug discovery as well as other basic scientific researches. SNP detection is basically the detection of a mutation in the genome. In a typical SNP array there is a quartet of probes which are usually 25 bases long. The middle base, the 13^{th} base, is either A, or C, or G, or T, with the rest of the sequences identical. Only one of the four probes, the one with the perfect match, will hybridize with the target fragment and yield the brightest signal. The 13^{th} base of this probe indicates the identity of that base in the genome.

In view of the above, there are aspects of the microarray technology that can be further improved. The sample for a microarray assay needs to be labeled and amplified in separate steps before hybridization. And since the amplification is a conventional end-point PCR, the original differences of the target may already be lost after this conventional end-point PCR amplification. Also, low-abundance targets may not be amplified with this conventional end-point PCR, since the high-abundance targets will always compete for reagents, and as the reaction progresses, the high-abundance targets will become more and more dominant and eventually overshadow the low-abundance targets.

### SUMMARY OF THE INVENTION

A broad object of this invention is to differentiate the ratio of various targets in a sample in a more quantitative and thus more precise way by applying the real-time amplification concept to an array assay.

Another object of this invention is to simplify and facilitate an array assay by completely eliminating pre-assay manipulation of targets such as amplification and labeling.

Still another object of this invention is to allow detection of ultra-low-abundance targets by its capability of detecting a single copy of a target.

This invention utilizes the methodology of a real-time PCR that uses fluorescent reporter to allow monitoring the progress of the amplification, and combines with an array method that attaches different primers onto a solid surface. Particularly, this method attaches both the first and second primers to the surface to allow forward and reverse extension, while amplification progress is monitored real-time. No pre-assay manipulation of any kind of the target is needed, since the fluorescent reporters are labeled to the primers, not to the target, as in conventional array assay. The primers fluoresce after they are extended and in double-stranded structure. No pre-assay amplification of target is needed, since amplification and monitoring is integrated into one single step.

### BRIEF DESCRIPTION OF THE DRAWINGS

FIG. 1 illustrates a system for real-time monitoring of nucleic acid amplification utilizing fluorescent reporter-labeled primers attached to a surface in an array.

FIG. 2 illustrates the arrangement of the first and second primers in a feature of a nucleic acid detection system wherein the first primers and the second primers are paired by a linker.

FIG. 3 illustrates the structure of the planar surface of a nucleic acid detection system with a plurality of hexagon zones with identical content and position of features.

FIG. 4 illustrates the fluorescent reporting means of extension of the first and second primers within a feature of a nucleic acid detection system.

FIG. 5 illustrates the sequencing procedure of a nucleic acid detection system with a set of four first primers with A, G, C, and T nucleotide at their 3′-ends, respectively.

### DETAILED DESCRIPTION OF THE INVENTION

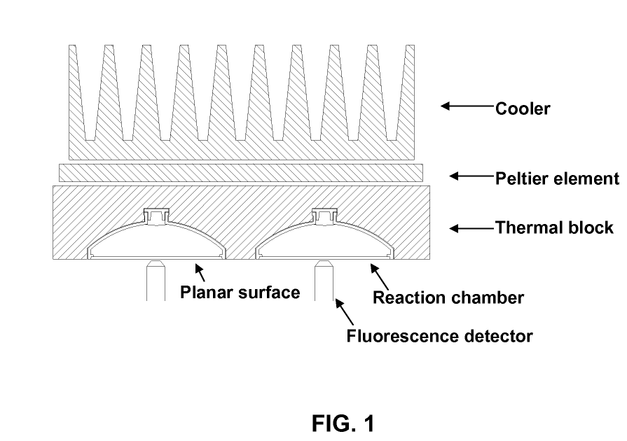

This invention provides a system that allows a real-time PCR of a microarray chip. As depicted in FIG. 1, this system comprises a thermal block that is attached with a Peltier element to adjust the temperature of the thermal block. The Peltier element is currently used in every PCR machine for rapid and accurate temperature adjusting. A specific reaction chamber is designed for this system. The reaction chamber comprises a planar surface as its bottom. This planar surface is made of a material, such as glass, to facilitate transmission of fluorescent light. The reaction chamber sits in a cavity in the thermal block with the planar surface facing outside for real-time monitoring. The reaction chamber has a kettle-like shape with a small lid, to maximize the planar surface area and minimize the reaction volume. This shape also gives a sufficient contact area between the reaction chamber and the thermal block, which is crucial for rapid temperature adjusting. For perfect contact of the reaction mixture with the planar surface the whole system is set in an inverted position, and the fluorescent detector is below the planar surface.

The basic element of this invention is the addressable features to which the first and second primers are attached. While the sequences of the first primers can be the ones along a genome, the sequences of the second primers are set such that they hybridize with the extension of their first primers along the genome. Multiple copies of a specific first primer and their second primers form a feature, and every feature is addressable.

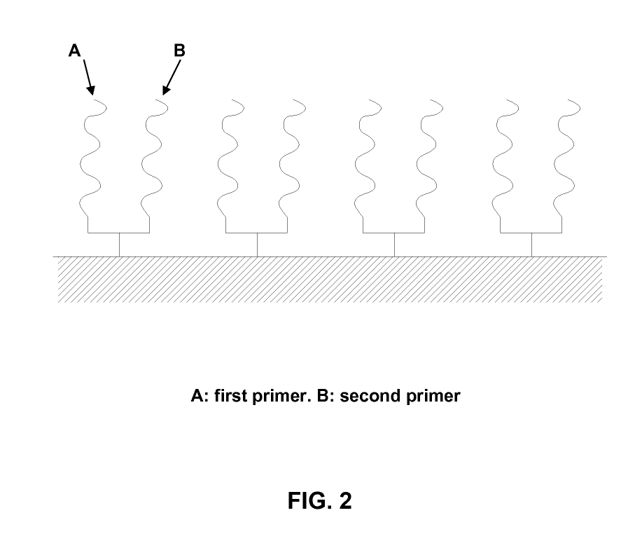

To attach the first and second primers to a feature a lithographic process is employed. In this process the first and second primers are first linked to a linker that has a first linking arm and a second linking arm, to which the first and second primers are linked, respectively, as depicted in FIG. 2. This compound of the primer pair is then linked to the chemical on the planar surface that is de-protected through the lithographic process. To form a pattern on the planar surface a lithographic mask is used, which has an array of micron-ranged pores. A light beam is projected to the lithographic mask to de-protect the chemical. After removing the mask, the compounds of the primer pair are added locally to the de-protected features, with some protected features between them. This lithographic process is repeated until all the features are linked with the primer pair compound.

This specific lithographic process is critical to this invention since in every feature the first and second primers must be evenly alternatively arranged. It is technically difficult to link the first and the second primers separately onto a feature, since the maximal distance between the first and the second primers should be within nanometer range. Therefore the seemingly only alternative is to link the primer pair compound to the features, which are within micron range.

To start an assay of this invention, a reaction mixture is added to the reaction chamber which comprises thermal-stable polymerases. When a target is added to the reaction chamber, it will hybridize with one of the first primers, and an extension will occur. A second primer surrounding the first primer will hybridize with this first primer extension, and prime a second primer extension. Now both the first primer extension and the second primer extension can be hybridized by surrounding second primer and first primer, respectively, and new extensions will occur. This first and second primer extension can cycle itself as the reaction continues. This is a unique feature of this invention, that is, even with a single hybridization of a target, the first and the second primer extension can repeat itself until all the first and second primer in a feature are extended.

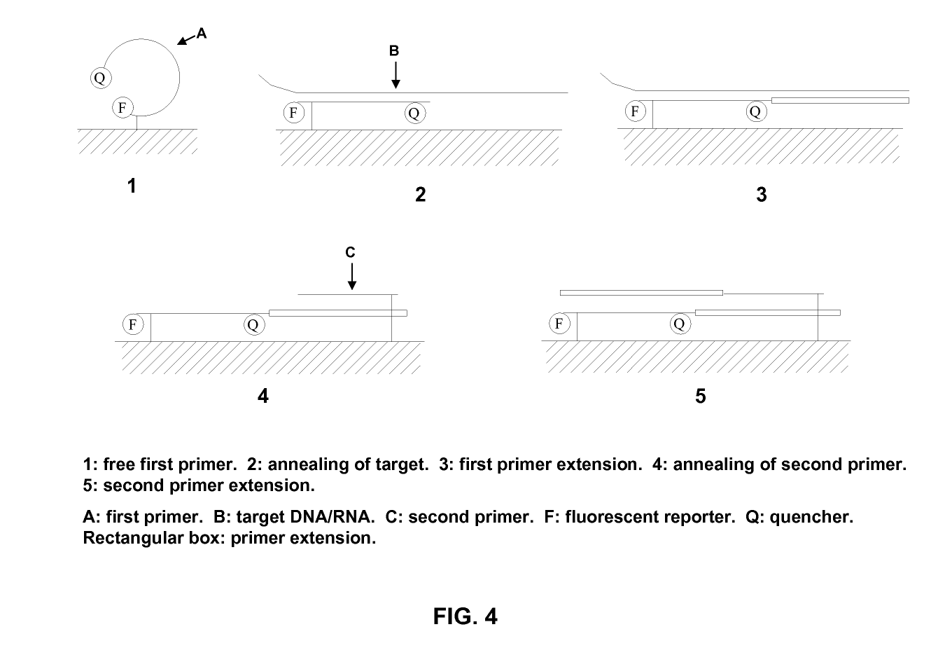

This invention uses three different fluorescent reporting methods to report extension of the first primer and the second primer. Firstly, the first primers and/or the second primers can be labeled with a fluorescence reporter and a quencher. Usually the fluorescence reporter and the quencher are labeled to either end of a primer, e.g. a fluorescence reporter at 5′-end and a quencher at 3′-end. A quencher can reduce the fluorescence of the fluorescence reporter by fluorescence resonance energy transfer (FRET) when the two moieties are separated by less than 100 angstroms. Therefore the fluorescence reporter will be quenched by the quencher when the primer is not extended. However, when the primer is extended and in double-stranded form, the quencher will usually be separated from the fluorescence reporter by more than 100 angstroms, thus unable to quench the fluorescence reporter. This fluorescent reporting method is depicted in FIG. 4.

Alternatively, primers labeled only with fluorescence reporters and without quenchers are designed for real-time PCR. The LUX™ primer from invitrogen corporation (Carsbad, Calif.) is such one. In this primer a fluorescent reporter is labeled to the 3′-end of the primer, and an extra 4-6 bases are attached to the 5′-end of the primer such that this extra stretch of 4-6 bases can hybridize with the 3′-end of the primer to form a hairpin structure. In the single-stranded form the fluorescent reporter in the primer has the least fluorescent intensity. However, when the primer is extended and in a double-stranded form, the fluorescent reporter has the most fluorescent intensity.

Thirdly, the primer extension can also be monitored with intercalating fluorescent dyes that bind to double-stranded DNA and fluoresce. One such commonly used dye in real-time PCR is SYBR™ from Molecular Probes (Eugene, Oreg.).

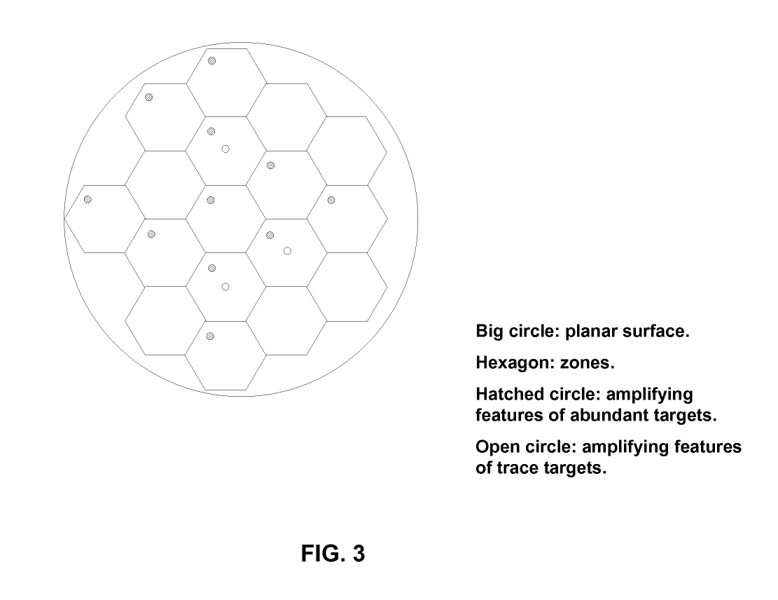

In order for accurate target quantitation, the planar surface is divided into a plurality of hexagon zones, as depicted in FIG. 3. There are approximately 20 zones in the planar surface of a one-centimeter-radius circle. In every zone there is one copy for every feature, and these features are in the same position in every zone. Therefore every zone is identical in terms of content and position of features. This multiple-zone configuration greatly increases the chance that every copy of a target hybridizes with its specific feature, since the targets spread out evenly in the reaction mixture above the planar surface.

Quantitation of a target is done by real-time measuring and combining the fluorescent intensity of all the copies of its feature. For an abundant target, a feature may be hybridized by more than one copy of the target, or the same feature may be hybridized by the target repeatedly during the reaction process. In both cases increased fluorescent intensity will reflect more hybridization.

This invention allows direct reverse transcription of transcripts when total RNAs are added directly into the reaction chamber. In the first step of this assay a transcript will hybridize with a first primer. After this hybridization a reverse transcription can occur with the reverse transcriptase in the reaction mixture. Once the cDNA fragment (which is actually the first primer extension) is generated the assay can proceed to completion even the original transcripts and/or the reverse transcriptase are degraded after this step. Since as little as one copy of a transcript can be detected this invention allows capturing virtually every transcript there is in a sample without any manipulation of the total RNA (in a tiling array).

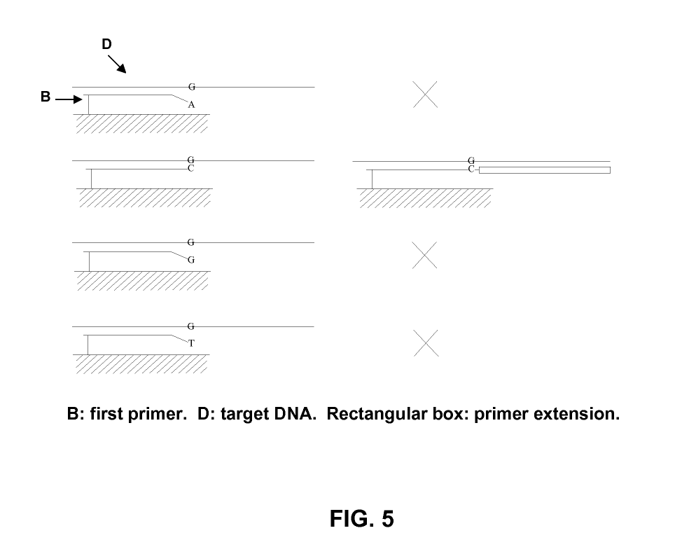

This invention utilizes a novel method for SNP detection and other resequencing purposes. Instead of using a quartet of oligomers with centering base of A, G, C, and T, respectively, this invention uses a quartet of primers with 3′-end base of A, G, C, and T, respectively. Only the primer with a matching 3′-end base with the target will be extended and recorded. Again, this invention allows direct addition of genomic DNA to the reaction chamber without any pre-assay manipulation. This novel SNP detection and resequencing method is depicted in FIG. 5.

Being an all solid state extension this invention has numerous advantages. Firstly, there is no need of any pre-assay manipulation and amplification. Targets can be added directly, no matter they are total RNA or genomic DNA, and no matter how small amount the targets are. No sample labeling, and no sample amplification is needed. Secondly, there is no amplification product released to the reaction mixture, and the targets never get modified or amplified, they are only counted. Actually samples are unchanged after the assay such that they can be re-used for another assay or for other purposes. Thirdly, this invention allows detection of single-copy targets that co-exist with other abundant targets. This is because for every target, no matter how abundant or how trace, there are same number of copies of features for them. The features for the abundant targets will be extended early. But once they are extended, there is no more extension occurring in these features. This means no competing for reagent, and no interrupting for extension of the features for the single-copy targets, no matter how long it will take for these features to be detected. Only in this solid state extension these single-copy targets can be detected, which are usually lost in other conventional detecting systems.

I claim:

1. A method of detecting target nucleic acids, said method comprising, (1) providing a reaction system comprising, (a) a thermal block, (b) a temperature-adjusting means attached to said thermal block, (c) at least one cavity on said thermal block, and (d) a fluorescence detector above said cavity, (2) providing a reaction chamber comprising, (A) a planar surface comprising, (a) a plurality of addressable features, each said addressable feature comprising (I) a plurality of first primers attached to said addressable feature, and (II) a plurality of second primers attached to said addressable feature, said second primer being able to hybridize with the extension from said first primer using said target nucleic acids as template, and (b) a plurality of copies of each said addressable feature evenly distributed on said planar surface, every said addressable feature having about same number of copies, (B) a reaction mixture in said reaction chamber, said reaction mixture comprising thermal-stable polymerases, and (C) a fluorescent reporting means in said reaction chamber such that said fluorescent reporting means can report extension of said first primers and/or said second primers, (3) adding said target nucleic acids to said reaction chamber, said target nucleic acids being able to hybridize with said first primers, (4) positioning said reaction chamber to said cavity such that said planar surface faces said fluorescence detector, (5) performing an amplification cycle using said temperature-adjusting means, and (6) quantitating said target nucleic acids with real-time measuring of extension of said first primers and said second primers.

2. A method of claim 1, wherein quantitating said target nucleic acids is by real-time measuring and combining the fluorescent intensity of every copy of said addressable features for a specific said target nucleic acid.

3. A method of claim 1, wherein a plurality of linkers is attached to said addressable feature, said linker comprising a first linking arm and a second linking arm, with a said first primer linked to said first linking arm, and a said second primer linked to said second linking arm.

4. A method of claim 1, wherein there are more than five copies of each said addressable feature on said planar surface with an area of approximately three square centimeters.

5. A method of claim 1, wherein said first primers are distributed approximately evenly along a genome.

6. A method of claim 1, wherein said first primer comprises a set of four primers with sequences only different at the 3′-end being A, C, G, and T, respectively.

7. A method of claim 1, wherein said target nucleic acids comprise total RNA, and said reaction mixture comprises reverse transcriptase.

8. A method of claim 1, wherein said fluorescent reporting means is an intercalating fluorescent dye in said reaction mixture.

9. A method of claim 1, wherein said fluorescent reporting means is a fluorescent reporter attached to said first primer and/or said second primer.

10. A method of claim 9, wherein a fluorescent quencher is attached to said first primer and/or said second primer.

11. A method of claim 1, wherein said planar surface is made of material facilitating transmission of fluorescent light,

12. A method of detecting target nucleic acids, said method comprising, (1) providing a reaction system comprising, (a) a thermal block, (b) a temperature-adjusting means attached to said thermal block, (c) at least one cavity on said thermal block, and (d) a fluorescence detector above said cavity, (2) providing a reaction chamber comprising, (A) a planar surface comprising a plurality of zones, said zones comprising, (a) a plurality of addressable features, each said addressable feature comprising, (I) a plurality of first primers attached to said addressable feature, and (II) a plurality of second primers attached to said addressable feature, said second primer being able to hybridize with the extension from said first primer using said target nucleic acids as template, and (b) one copy of a specific said addressable feature in every said zone, said copies of a specific said addressable feature being evenly distributed in said planar surface, (B) a reaction mixture in said reaction chamber, said reaction mixture comprising thermal-stable polymerases, and (C) a fluorescent reporting means in said reaction chamber such that said fluorescent reporting means can report extension of said first primers and/or said second primers, (3) adding said target nucleic acids to said reaction chamber, said target nucleic acids being able to hybridize with said first primers, (4) positioning said reaction chamber to said cavity such that said planar surface faces said fluorescence detector, (5) performing an amplification cycle using said temperature-adjusting means, and (6) quantitating said target nucleic acids with real-time measuring of extension of said first primers and/or said second primers.

13. A method of claim 12, wherein quantitating said target nucleic acids is by real-time measuring and combining the fluorescent intensity of every copy of said addressable features for a specific said target nucleic acid.

14. A method of claim 12, wherein a plurality of linkers is attached to said addressable feature, said linker comprising a first linking arm and a second linking arm, with a said first primer linked to said first linking arm, and a said second primer linked said second linking arm.

15. A method of claim 12, wherein there are more than five of said zones on said planar surface with an area of approximately three square centimeters.

16. A method of claim 12, wherein said first primers are distributed approximately evenly along a genome.

17. A method of claim 12, wherein said first primer comprises a set of four primers with sequences only different at the 3′-end being A, C, G, and T, respectively.

18. A method of claim 12, wherein said target nucleic acids comprise total RNA, and said reaction mixture comprises reverse transcriptase.

19. A method of claim 12, wherein said fluorescent reporting means is an intercalating fluorescent dye in said reaction mixture.

20. A method of claim 12, wherein said fluorescent reporting means is a fluorescent reporter attached to said first primer and/or said second primer.

21. A method of claim 20, wherein a fluorescent quencher is attached to said first primer and/or said second primer.

22. A method of claim 12, wherein said planar surface is made of material facilitating transmission of fluorescent light.

23. A method for fabricating a nucleic acid array, said method comprising, (1) providing a lithographic mask comprising an array of addressable pores, (2) providing a planar surface, (3) providing a chemical sprayed onto said planar surface, said chemical comprising a photo-labile protecting group, (4) forming a pattern of a plurality of addressable features on said planar surface by projecting a light beam onto said planar surface covered with said lithographic mask to de-protect said chemical, (5) providing a nucleic acid primer compound comprising, (a) a linker, (b) a first linking arm attached to said linker, (c) a second linking arm attached to said linker, (d) a first nucleic acid primer attached to said first linking arm, and (e) a second nucleic acid primer attached to said second linking arm, said second nucleic acid primer being able to hybridize with the extension from said first nucleic acid primer using a template, (6) removing said lithographic mask from said planar surface, and (7) adding locally said nucleic acid primer compound onto de-protected said addressable feature to link said linker to de-protected said chemical.
